(19) United States
(12) Patent Application Publication
(10) Pub. No.: US 2023/0263834 A1
(43) Pub. Date: Aug. 24, 2023

(54) METHOD OF PREVENTING, REDUCING OR DELAYING FATTY LIVER DISEASE

(71) Applicant: Abbott Laboratories, Abbott Park, IL (US)

(72) Inventors: José María LÓPEZ PEDROSA, Cenes de la Vega (ES); Ricardo RUEDA CABRERA, Granada (ES); Jorge GARCÍA MARTÍNEZ, Armilla (ES)

(21) Appl. No.: 18/006,765

(22) PCT Filed: Jul. 26, 2021
(86) PCT No.: PCT/US2021/043209

§ 371 Date: Jan. 25, 2023

(30) Foreign Application Priority Data
Jul. 31, 2020 (EP) 20382704.3

(51) Int. Cl. A61K 35/20; A61P 1/16; A61K 9/16

(52) U.S. Cl. CPC A61K 35/20; A61P 1/16; A61K 9/16

(57) ABSTRACT

A method of preventing, reducing, or delaying the onset of fatty liver disease in a subject comprises enterally administering intact bovine milk-derived exosomes consisting of endogenous cargo to a subject in need thereof in an amount effective to reduce hepatic lipid accumulation. In a specific embodiment, the intact bovine milk-derived exosomes consisting of endogenous cargo can be administered in an amount effective to reduce de novo lipogenesis in the subject. The intact bovine milk-derived exosomes consisting of endogenous cargo can be administered to the subject directly or in a nutritional composition.

### FIELD OF THE INVENTION

The present invention relates to methods of preventing, reducing, or delaying the onset of fatty liver disease in a subject by administering intact bovine milk-derived exosomes.

### BACKGROUND OF THE INVENTION

The incidence of metabolic diseases has increased significantly worldwide in the last few decades, in large part as a result of sedentary lifestyles and unhealthy eating patterns. Non-alcoholic fatty liver disease (NAFLD), which has been linked to obesity and type 2 diabetes, has been predicted to be a global epidemic. NAFLD represents a wide spectrum of diseases that originate with excess accumulation of fat within the liver, or hepatic steatosis, and is associated with multiple detrimental effects, including increased mortality due to liver failure, cardiovascular disease, and hepatocellular carcinoma. Contos M J, Sanyal A J. The Clinicopathologic Spectrum and Management of Nonalcoholic Fatty Liver Disease. Adv Anat Pathol 2002; 9(1): 37-51.

NAFLD, which is currently considered as a component of the metabolic syndrome, is an increasingly common health concern in both children and adults. In fact, it has been reported that NAFLD is the most common liver disease in the world, affecting up to one fourth of the population. See Ipsen D H, Lykkesfeldt J, Tveden-Nyborg P. Molecular mechanisms of hepatic lipid accumulation in non-alcoholic fatty liver disease. Cell Mol Life Sci 2018; 75(18): 3313-3327; Preiss D, Sattar N. Non-alcoholic fatty liver disease: an overview of prevalence, diagnosis, pathogenesis and treatment considerations. Clin Sci (Lond) 2008; 115(5): 141-150; Yu E L, Golshan S, Harlow I C E, Angeles J E, Durelle J, Goyal N P et al. Prevalence of nonalcoholic fatty liver disease in children with obesity. Pediatr 2019; 207: 64-70. Regional prevalence rates are highest in the Middle East and South America and lowest in Africa, and the incidence of NAFLD among severely obese and type 2 diabetic patients has been estimated at about 90% and 75%, respectively. Remarkably, even lean and otherwise healthy individuals have been reported to develop NAFLD. In fact, the prevalence of NAFLD has made NAFLD the second most common cause of liver transplantation in the United States. See Ipsen D H et al.; Mikolasevic, Ivana, et al. “Nonalcoholic Fatty Liver Disease and Liver Transplantation—Where Do We Stand?” World Journal of Gastroenterology, vol. 24, no. 14, 2018, pp. 1491-1506.

Unfortunately, there is currently neither an NAFLD-specific therapeutic agent available in the market nor a generally accepted NAFLD treatment. To date, the most effective treatment for NAFLD consists of implementing lifestyle changes aimed at reducing weight and increasing exercise. In addition, and given that NAFLD seems to be worsened by insulin resistance, insulin sensitizers such as pioglitazone, a thiazolidinedione compound, have been used. However, the therapeutic use of drugs that restore insulin sensitivity have raised some concerns regarding increased cardiovascular risks and other undesired side-effects. There is therefore an urgent need to develop new therapeutic strategies to fight NAFLD.

Accordingly, methods of preventing, reducing, or delaying the onset of NAFLD are desirable.

### SUMMARY OF THE INVENTION

It is therefore an object of the invention to provide methods which prevent, reduce, or delay the onset of fatty liver disease.

In one embodiment, the invention is directed to a method of preventing, reducing, or delaying the onset of fatty liver disease in a subject, comprising administering intact bovine milk-derived exosomes consisting of endogenous cargo to a subject in need thereof in an amount effective to reduce hepatic lipid accumulation.

In a more specific embodiment, the invention is directed to a method of preventing, reducing, or delaying the onset of fatty liver disease in a subject, comprising administering intact bovine milk-derived exosomes consisting of endogenous cargo to a subject in need thereof in an amount effective to reduce hepatic lipid accumulation, wherein the intact bovine milk-derived exosomes consisting of endogenous cargo are administered to a subject in a nutritional composition.

The methods of preventing, reducing, or delaying the onset of fatty liver disease in a subject of the present invention are advantageous in that they provide a convenient therapeutic strategy for preventing and/or combatting fatty liver disease, particularly NAFLD. This and additional objects and advantages of the invention will be more fully apparent in view of the following detailed description.

### BRIEF DESCRIPTION OF THE DRAWINGS

The drawings are illustrative of certain embodiments of the invention and exemplary in nature and are not intended to limit the invention defined by the claims, wherein:

FIG. 1 illustrates Fatty acid synthase (FAS) protein levels of HEPG2 cells treated with both intact bovine milk-derived exosomes and sonicated exosomes, as described in Example 2.

FIG. 2 illustrates FAS protein levels in rat models supplemented with bovine milk-derived exosomes and control rat models that did not receive bovine milk-derived exosomes, as described in Example 3.

FIG. 3 illustrates FAS gene expression in the livers of rat models supplemented with bovine milk-derived exosomes and control rat models that did not receive bovine milk-derived exosomes, as described in Example 3.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain embodiments of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “bovine milk-derived exosomes” as used herein, unless otherwise specified, refers to exosomes that have been substantially separated from other bovine milk components such as lipids, cells, and debris, and are concentrated in an amount higher than that found in bovine milk. The exosomes are small, extracellular vesicles and account for a minor percentage of milk's total content. In specific embodiments, the isolated intact exosomes are provided in a liquid or powdered exosome-enriched product which also contains co-isolated milk solids. In one embodiment, the isolated exosomes are provided in a product that contains at least 10 wt % exosomes, at least 15 wt % exosomes, at least 20 wt %, or at least 25 wt % exosomes, and a balance of other bovine milk-isolated components.

The term “endogenous cargo” as used herein refers to bioactive agents, therapeutics (e.g. miRNA), and/or other biomolecules which are inherently present in a bovine milk-derived exosome, for example functional lipids, proteins, and miRNAs. The term “exogenous cargo” as used herein refers to bioactive agents, therapeutics, and/or other molecules or biomolecules that are not inherently present within a bovine milk-derived exosome, but have instead been loaded into, added, or included in the exosome, for example via methods such as electroporation, lipofection, sonication, and calcium chloride. The term “exosomes consisting of endogenous cargo” thus refers to exosomes having no exogenous cargo, i.e., exosomes in which no content has been loaded into, added, or included in the exosomes in any way by any outside means, for example through electroporation, lipofection, sonication, or calcium chloride.

The term “enterally” or “enteral administration” as used herein refers to administration involving the esophagus, stomach, and small and large intestines (i.e., the gastrointestinal tract). Examples of enteral administration include oral, sublingual, and rectal administration.

The term “intact exosomes” as used herein refers to exosomes in which the vesicle membrane is not ruptured and/or otherwise degraded and the endogenous cargo, i.e., the bioactive agents, therapeutics (e.g. miRNA), and/or other biomolecules which are inherently present in a bovine milk-derived exosome, are retained therein in active form.

The liver has a crucial role in lipid metabolism in that it is responsible for the synthesis of new fatty acids, their export to other tissues, and their utilization as energy substrates. Hepatic lipid accumulation occurs when triglyceride production and uptake into the liver exceeds clearance or removal. The liver acquires lipids through two main pathways: the uptake of circulating fatty acids (either dietary fatty acids or non-esterified fatty acids resulting from increased lipolysis of peripheral fat depots) and via de novo lipogenesis (DNL). Hepatic lipids are removed through β-oxidation in the mitochondria and through their export as very low density lipoproteins (VLDLs). As touched on above, overeating, obesity, insulin resistance and type 2 diabetes are among the conditions that result in hepatic lipid accumulation.

DNL is a highly regulated pathway that enables liver to convert excess carbohydrates into fatty acids, which are ultimately esterified with glycerol-3-phosphate to yield triglycerides. Fatty acid synthase (FAS) catalyzes the last step in fatty acid biosynthesis and it is therefore thought to be a key determinant of the maximal hepatic capacity to generate fatty acids through the DNL pathway.

As indicated above, the present invention provides methods of preventing, reducing, or delaying the onset of fatty liver disease. Without wishing to be bound by any particular theory, the methods of the present invention prevent, reduce, or delay the onset of fatty liver disease, for example NAFLD, by reducing hepatic lipogenesis via administration of bovine milk-derived exosomes to a subject in need thereof. The present inventors have surprisingly demonstrated that intact bovine milk-derived exosomes consisting of endogenous cargo are unexpectedly capable of downregulating the expression of FAS.

In one embodiment, a method of preventing, reducing, or delaying the onset of fatty liver disease in a subject is provided. The method comprises administering intact bovine milk-derived exosomes consisting of endogenous cargo to a subject in need thereof in an amount effective to reduce hepatic lipid accumulation.

By way of example, the dosage of bovine milk-derived exosomes is from about 0.01 to about 10 g of exosomes, which may be administered directly or via addition to a nutritional composition, as discussed below. More specifically, the dosage of bovine milk-derived exosomes may be from about 0.1 to about 10 g, from about 0.1 to about 5 g, or from about 1 to about 5 g, administered directly or via addition to a nutritional composition. In further embodiments, the bovine milk-derived exosomes can be administered to a subject from about 1 to about 6 times per day or per week, or from about 1 to about 5 times per day or per week, or from about 1 to about 4 times per day or per week, or from about 1 to about 3 times per day or per week.

Generally, the exosomes are obtained from a whey-containing bovine milk fraction using gentle procedures which do not disrupt the exosome vesicle membrane, thereby leaving the exosomes intact and active bioactive agents contained within the exosome structure.

Various methods may be employed to isolate exosomes with care being exercised to avoid disruption of the lipid membrane. Fresh bovine milk, refrigerated bovine milk, thawed frozen bovine milk, or otherwise preserved bovine milk, or any bovine milk fraction containing exosomes, for example, cheese whey, may be employed as a source of exosomes. In specific embodiments, isolating the exosomes comprises performing the isolation immediately upon obtaining milk from a bovine. In additional embodiments, isolating the exosomes comprises performing the isolation within about 1 day, or about 2 days, or about 3 days, or about 4 days, or about 5 days or about 6 days, or about 7 days from the time of obtaining the milk from a bovine. In specific embodiments, the exosomes are isolated within about 10 days, or within about 14 days from the time of obtaining milk from a bovine. In additional embodiments, the bovine milk may be frozen and then thawed for processing for isolating exosomes, with the bovine milk preferably having been frozen within about 1 day, or about 2 days, or about 3 days, or about 4 days, or about 5 days or about 6 days, or about 7 days from the time of obtaining the milk from a bovine. Thawed milk is preferably processed immediately upon thawing. In a specific embodiment, fresh bovine milk is subjected to the processing within about 5 days of obtaining the milk from a bovine, or thawed bovine milk which is subjected to processing is thawed from bovine milk that was frozen within about 5 days of obtaining the milk from a bovine.

In one embodiment, a whey-containing bovine milk fraction or, specifically, cheese whey, serves as a source of exosomes. In a specific embodiment, the whey-containing bovine milk fraction is provided by lowering the pH of a bovine milk product, for example, to about 3.0 to 4.6, to precipitate milk solids, and removing the milk solids. Such a fraction is often produced as a byproduct in cheese-making and is referred to as cheese whey.

By way of example, a gentle procedure of obtaining intact bovine milk-derived exosomes may comprise centrifugation at specific speeds, times and/or temperatures. The bovine milk, which is optionally frozen and subsequently thawed, is centrifuged a first time, which separates the milk into an upper fraction (i.e., lipid fraction top layer), a middle fraction (i.e., whey middle fraction), and a first pellet of cells and debris. The middle fraction is then centrifuged, ideally two times and at a speed faster than the speed of the first centrifugation, to remove residual fat and other debris. The resulting concentrated clear whey fraction can be filtered to remove residual debris. The resulting filtrate is then centrifuged, ideally at a speed faster than the speed of the second centrifugation, to produce a third pellet containing exosomes. The exosomes can then be resuspended, frozen, and optionally freeze-dried. A more detailed example of isolating intact exosomes from bovine milk is set forth in Example 1 below.

In a specific embodiment of the invention, the intact bovine milk-derived exosomes consisting of endogenous cargo are administered in an amount effective to reduce de novo lipogenesis in the subject.

In another specific embodiment, the fatty liver disease is NAFLD. In a further specific embodiment, the subject is suffering from non-alcoholic fatty liver disease (NAFLD). Nonalcoholic fatty liver disease comprises simple fatty liver disease, which is also referred to as nonalchoholic fatty liver (NAFL) or isolated fatty liver, and nonalcoholic steatohepatitis (NASH). Simple fatty liver disease is a form of NAFLD wherein there is fat in the liver, but there is little or no inflammation or liver cell damage. NASH, which is a more severe progression of NAFLD, is characterized by hepatic inflammation, hepatocyte damage, and/or liver fibrosis, which increases with the progression of the disease and may cause cirrhosis and hepatocellular carcinoma.

In a specific embodiment, the intact bovine milk-derived exosomes consisting of endogenous cargo are administered orally.

In one specific embodiment, the intact bovine milk-derived exosomes consisting of endogenous cargo are administered directly to the subject, for example, in a dry powder form or suspended in a liquid. In another specific embodiment, the intact bovine milk-derived exosomes consisting of endogenous cargo are administered to the subject in a nutritional composition. In a further specific embodiment, the nutritional composition is in the form of a powder. In another specific embodiment, the nutritional composition is in the form of a liquid.

When the nutritional composition is in the form of a liquid, for example, reconstituted from a powder or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

When the nutritional composition is a powder, for example, a serving size is from about 40 g to about 60 g, such as 45 g, or 48.6 g, or 50 g, to be administered as a powder or to be reconstituted in from about 1 ml to about 500 ml of liquid, such as about 225 ml, or from about 230 ml to about 245 ml.

In a specific embodiment, the nutritional composition is administered orally. By way of example, the nutritional composition can be administered to a subject from about 1 to about 6 times per day or per week, or from about 1 to about 5 times per day or per week, or from about 1 to about 4 times per day or per week, or from about 1 to about 3 times per day or per week.

In another embodiment, the nutritional composition further comprises protein, carbohydrate, and/or a fat.

A wide variety of one or more proteins, carbohydrates, and/or fats can be used in the nutritional composition of the invention. For example, the protein can include intact, hydrolyzed, and/or partially hydrolyzed protein, which can be derived from a suitable source such as milk (e.g., casein, whey), animal (e.g., meat, fish), cereal (e.g., rice, corn), vegetable (e.g., soy, pea), and combinations thereof. In a specific embodiment, the protein comprises whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hamp proteins, flax seed proteins, earthworm protein, insect protein, or combinations of two or more thereof. The protein may also include one or a mixture of amino acids (often described as free amino acids) known for use in nutritional products, and/or metabolites thereof, or a combination of one or more such amino acids and/or metabolites, with the intact, hydrolyzed, and partially hydrolyzed proteins described herein. The amino acids may be naturally occurring or synthetic amino acids. In one embodiment, one or more branched chain amino acids (leucine, isoleucine and/or valine) and/or one or more metabolites of branched chain amino acids, for example, leucic acid (also known as α-hydroxyisocaproic acid or HICA), keto isocaproate (KIC), and/or β-hydroxy-β-methylbutyrate (HMB), are included as a protein in the nutritional compositions.

The nutritional composition may comprise protein in an amount from about 1 wt % to about 30 wt % of the nutritional composition. More specifically, the protein may be present in an amount from about 1 wt % to about 25 wt % of the nutritional composition, including about 1 wt % to about 20 wt %, about 2 wt % to about 20 wt %, about 1 wt % to about 15 wt %, about 1 wt % to about 10 wt %, about 5 wt % to about 10 wt %, about 10 wt % to about 25 wt %, or about 10 wt % to about 20 wt % of the nutritional composition. Even more specifically, the protein comprises from about 1 wt % to about 5 wt % of the nutritional composition, or from about 20 wt % to about 30 wt % of the nutritional composition.

In another specific embodiment, the carbohydrate comprises human milk oligosaccharides (HMOs), maltodextrin, hydrolyzed starch, glucose polymers, corn syrup, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, inulin, fructooligosaccharides, or combinations of two or more thereof.

The nutritional composition may comprise carbohydrate in an amount from about 5 wt % to about 75 wt % of the nutritional composition. More specifically, the carbohydrate may be present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

In a further embodiment, the fat comprises coconut oil, fractionated coconut oil, soy oil, corn oil, olive oil, safflower oil, medium chain triglyceride oil (MCT oil), high gamma linolenic (GLA) safflower oil, sunflower oil, palm oil, palm kernel oil, palm olein, canola oil, marine oils, fish oils, algal oils, borage oil, cottonseed oil, fungal oils, at least one omega-3 fatty acid, interesterified oils, transesterified oils, structured lipids, and combinations of two or more thereof. In a specific embodiment, the at least one omega-3 fatty acid of the composition is selected from the group consisting of eicosapentaenoic acid, docosahexaenoic acid, arachidonic acid, and alpha-linolenic acid.

The nutritional composition may comprise fat in an amount of from about 0.5 wt % to about 30 wt % of the nutritional composition. More specifically, the fat may be present in an amount from about 0.5 wt % to about 10 wt %, about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

The concentration and relative amounts of the sources of protein, carbohydrate, and fat in the exemplary nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition, and, more specifically, such composition is in liquid form. In another specific embodiment, the nutritional composition comprises a source of protein in an amount of about 10 wt % to about 25 wt %, a source of carbohydrate in an amount of about 40 wt % to about 70 wt %, and a source of fat in an amount of about 5 wt % to about 20 wt %, based on the weight of the nutritional composition, and, more specifically, such composition is in powder form.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

In a specific embodiment, the nutritional composition comprises protein, carbohydrate, fat, and one or more nutrients selected from the group consisting of vitamins, minerals, and trace minerals. Non-limiting examples of vitamins include vitamin A, vitamin B12, vitamin C, vitamin D, vitamin E, vitamin K, thiamine, riboflavin, pyridoxine, niacin, folic acid, pantothenic acid, biotin, choline, inositol, and/or salts and derivatives thereof, and combinations thereof. Non-limiting examples of minerals and trace minerals include calcium, phosphorus, magnesium, zinc, manganese, sodium, potassium, molybdenum, chromium, iron, copper, and/or chloride, and combinations thereof.

The nutritional composition may also comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

In a further specific embodiment, the nutritional composition comprises about 0.001 to about 10 wt %, about 0.001 to about 9 wt %, about 0.001 to about 8 wt %, about 0.001 to about 7 wt %, about 0.001 to about 6 wt %, about 0.001 to about 5 wt %, about 0.001 to about 4 wt %, about 0.001 to about 3 wt %, about 0.001 to about 2 wt %, about 0.001 to about 1 wt %, about 0.01 to about 10 wt %, about 0.01 to about 9 wt %, about 0.01 to about 8 wt %, about 0.01 to about 7 wt %, about 0.01 to about 6 wt %, about 0.01 to about 5 wt %, about 0.01 to about 4 wt %, about 0.01 to about 3 wt %, about 0.01 to about 2 wt %, about 0.01 to about 1 wt %, about 0.1 to about 10 wt %, about 0.1 to about 9 wt %, about 0.1 to about 8 wt %, about 0.1 to about 7 wt %, about 0.1 to about 6 wt %, about 0.1 to about 5 wt %, about 0.1 to about 4 wt %, about 0.1 to about 3 wt %, about 0.1 to about 2 wt %, about 0.1 to about 1 wt %, about 1 to about 10 wt %, about 1 to about 9 wt %, about 1 to about 8 wt %, about 1 to about 7 wt %, about 1 to about 6 wt %, about 1 to about 5 wt %, about 1 to about 4 wt %, about 1 to about 3 wt %, about 1 to about 2 wt %, about 2 to about 10 wt %, about 2 to about 9 wt %, about 2 to about 8 wt %, about 2 to about 7 wt %, about 2 to about 6 wt %, about 2 to about 5 wt %, about 2 to about 4 wt %, about 2 to about 3 wt %, about 3 to about 10 wt %, about 3 to about 9 wt %, about 3 to about 8 wt %, about 3 to about 7 wt %, about 3 to about 6 wt %, about 3 to about 5 wt %, about 3 to about 4 wt %, about 4 to about 10 wt %, about 4 to about 9 wt %, about 4 to about 8 wt %, about 4 to about 7 wt %, about 4 to about 6 wt %, about 4 to about 5 wt %, about 5 to about 10 wt %, about 5 to about 9 wt %, about 5 to about 8 wt %, about 5 to about 7 wt %, about 5 to about 6 wt %, about 6 to about 10 wt %, about 6 to about 9 wt %, about 6 to about 8 wt %, about 6 to about 7 wt %, about 7 to about 10 wt %, about 7 to about 9 wt %, about 7 to about 8 wt %, about 8 to about 10 wt %, about 8 to about 9 wt %, or about 9 to about 10 wt % of the intact bovine milk-derived exosomes consisting of endogenous cargo, based on the weight of the nutritional composition.

In a further embodiment, the subject is a human.

The following Examples demonstrate various embodiments of the invention.

### EXAMPLES

### Example 1: Bovine Milk-Derived Exosomes

This example describes a method of isolating exosomes from bovine milk to provide an exosome-enriched product containing intact exosomes consisting of endogenous cargo. Upon reception at 4° C., raw, unprocessed milk was aliquoted and immediately frozen at −80° C. Aliquots were thawed on ice and centrifuged at 12,000 G for 15 minutes at 4° C. The upper fraction (i.e., lipid fraction top layer) and a first pellet of cells and debris were discarded and the middle fraction (i.e., whey middle fraction) was transferred to a clean tube. The whey middle fraction was centrifuged two times, each time at 21,500 G for 30 minutes at 4° C., to remove residual fat (upper layer) and other debris (second pellet). The concentrated clear whey fraction that was obtained from the second centrifugation of the whey middle fraction was filtered through a 0.22 micrometers (μm) polyethersulfone (PES) filter (hydrophilic, low protein retention) to remove residual debris. The resulting filtrate was ultracentrifuged at 100,000 G for 1 hour at 4° C. to produce a third pellet containing exosomes.

Following isolation, the exosomes were resuspended in either sterile PBS buffer (137 mM NaCl, 2.7 mM KCl, 8 mM Na₂HPO₄, and 2 mM KH₂PO₄; pH 7.4) or sterile water in a centrifugation tube. In order to dissolve the pellet without disrupting the milk exosome membrane, the sterile PBS buffer or water was added to the centrifugation tube and the pellet in buffer/water was incubated for 12-36 hours in an orbital shaker at 4° C. and 150 rpm. This allows suspension of the exosomes without disrupting the membrane. Once dissolved, the milk exosomes were frozen at −80° C. for at least 2 hours. The frozen products were subsequently freeze-dried in a Telstar Cryodos −80 freeze dryer at −80° C. and <0.3 mbar for at least 24 hours to 48 hours to provide a powdered exosome product. The frozen products were not thawed prior to freeze-drying.

Following the step of freeze-drying, a portion of the powdered exosomes was dissolved in either sterile PBS or water and placed in an Ultrasons P-selecta sonifier for 1 hour. Following sonication, these exosomes were incubated for 15 minutes at 95° C. in order to ensure complete disruption of the milk exosome membrane.

### Example 2: In Vitro Assay

This example demonstrates that intact bovine milk-derived exosomes are able to decrease the levels of FAS protein, a key element of the hepatic lipid biosynthetic pathway. This was shown by demonstrating an in vitro reduction in FAS protein expression in the human liver hepatoma cell line HEPG2 (ATCC® HB-8065™).

The HEPG2 cell line (ATCC® HB-8065™) was grown at 37° C. in Dulbecco's Modified Eagle's Medium (DMEM) supplemented with 10% (v/v) fetal bovine serum (FBS), 2 mM glutamine plus 100 U/mL penicillin, and 0.1 mg/mL streptomycin in an atmosphere of 5% CO₂ and 95% air, and was maintained at subconfluent densities in the growth media. The HEPG2 cells were incubated with 15 μg/mL of either the intact bovine milk-derived powdered exosome product from Example 1 (suspended in sterile water) or the bovine milk-derived powdered exosome product, sonicated as described in Example 1. After the incubation period, plates were flash frozen in liquid nitrogen and processed.

The HEPG2 cells were lysed with radioimmunoprecipitation (RIPA) buffer supplemented with protease inhibitors. Protein concentration was determined using the bicinchoninic acid method and 20-60 μg were loaded for western blot. The antibodies used included fatty acid synthase (FAS; Santa Cruz, Calif., USA) and glyceraldehyde-3-phosphate dehydrogenase (GAPDH; Sigma-Aldrich, Saint Louis, Mo., USA). GAPDH was used as a load control. Data were normalized by adjusting the values of untreated controls to 100%.

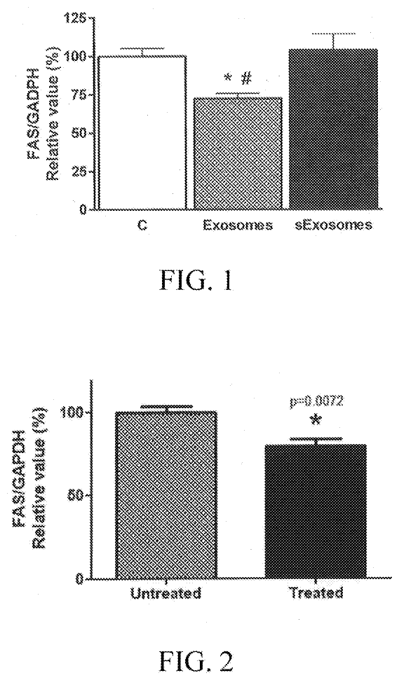

As evidenced by the results in FIG. 1, when compared to the untreated control (labeled “C” in FIG. 1), hepatic cells incubated with 15 μg exosome/mL of the intact bovine milk-derived exosomes (labeled “Exosomes” in FIG. 1) showed a statistically significant reduction in FAS protein expression. On the other hand, an equivalent amount of the sonicated exosomes (labeled “sExosomes” in FIG. 1) failed to decrease FAS protein levels. These results indicate that only the intact milk exosomes were able to decrease the levels of FAS protein, a key element of the hepatic lipid biosynthetic pathway.

### Example 3: In Vivo Assay

This example demonstrates that the consumption of intact bovine milk-derived exosomes are able to decrease FAS gene expression and FAS protein levels. This was shown by demonstrating an in vivo reduction in FAS gene expression and FAS protein expression in an animal model.

26 male Wistar rats (12 weeks old) were individually housed in cages and kept under 12 hour light-12 hour dark cycles. Room temperature was maintained at 21° C. Rats were randomly allocated to either a Treated group or a Control group and were fed a standard rodent diet (AIN93M) for 21 days. The Treated group was also supplemented with the intact milk powdered exosome product of Example 1 (141 mg/day of the powdered exosome product) resuspended in water. Rats that were allocated to the Control group received the same daily dose of water, however no exosomes were added. After 21 days, the rats were sacrificed and their livers were isolated and immediately preserved in liquid nitrogen in order to avoid tissue damage. Following isolation of the livers, mRNA was extracted from the frozen livers and was retrotranscribed into cDNA.

The effect of consuming intact bovine milk-derived exosomes on FAS protein levels in an in vivo animal model was analyzed. For protein analysis, liver lysates were obtained in lysis buffer (RIPA buffer containing protease inhibitors). Protein concentration was determined using the bicinchoninic acid method and 20/60 μg were loaded for western blot. The antibodies used included FAS (Santa Cruz, Calif., USA) and GAPDH (Sigma-Aldrich, Saint Louis, Mo., USA). GAPDH was used as a load control. Data were normalized adjusting the values of the untreated controls to 100%. As evidenced by the data shown in FIG. 2, rats that consumed intact bovine milk-derived exosomes showed a significant reduction in FAS protein levels as compared to rats that did not consume intact bovine milk-derived exosomes. This indicates that the consumption of intact bovine milk-derived exosomes reduces FAS protein levels. Further, the results illustrated in FIG. 2 indicate that the intact bovine milk-derived exosomes are capable of surviving the harsh conditions of the gastrointestinal tract and, as a result of this demonstrated decrease in FAS protein levels, are able to decrease hepatic lipogenesis. The consumption of intact bovine milk-derived exosomes can thus be used to prevent, reduce, or delay the onset of fatty liver disease, for example NAFLD, and other diseases or conditions that are associated with increased hepatic fat content.

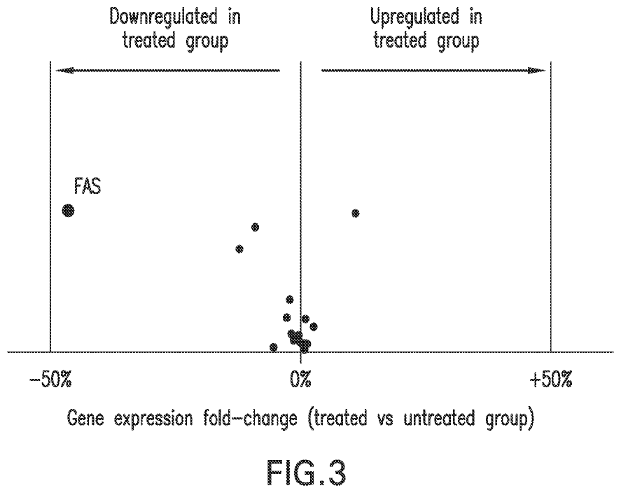

The effects of intact bovine milk-derived exosome consumption on FAS gene expression in an in vivo animal model was also analyzed. FAS gene expression levels were analyzed using the “insulin signaling pathway (030ZA)” Qiagen RT2 profiler array. As shown in FIG. 3, the Treated group, i.e., the rats that consumed intact bovine milk-derived exosomes for a feeding period of 21 days, exhibited a reduction of about 45% as compared to the Control group, i.e., rats that did not consume intact bovine milk-derived exosomes. Each dot in FIG. 3 represents a specific gene. As illustrated by the dots around the middle line in the figure, most hepatic genes were not upregulated or downregulated by the consumption of intact bovine milk-derived milk exosomes. However, FAS gene expression was significantly downregulated in the livers of rats supplemented with the intact bovine milk-derived exosomes. These results indicate that consumption of intact bovine milk-derived exosomes is able to reduce FAS gene expression. Further, the data shown in FIG. 3 indicates that intact bovine milk-derived exosomes are capable of surviving the harsh conditions of the gastrointestinal tract and, as a result of this demonstrated decrease in FAS gene expression, are able to decrease hepatic lipogenesis. The consumption of intact bovine milk-derived exosomes can thus be used to prevent, reduce, or delay the onset of fatty liver disease, for example NAFLD, and other diseases or conditions that are associated with increased hepatic fat content.

In summary, intact bovine milk-derived exosomes consisting of endogenous cargo decrease hepatic lipogenesis, which has a significant application in diseases or conditions that are associated with increased hepatic fat content, including fatty liver disease and more specifically NAFLD. The in vivo consumption of an effective dose of intact bovine milk-derived exosomes can promote a decrease in FAS gene expression and protein levels.

The specific embodiments and examples described herein are exemplary only and are not limiting to the invention defined by the claims.

1. A method of preventing, reducing, or delaying the onset of fatty liver disease in a subject, comprising:
enterally administering intact bovine milk-derived exosomes consisting of endogenous cargo to a subject in need thereof in an amount effective to reduce hepatic lipid accumulation.

2. The method of claim 1, wherein the intact bovine milk-derived exosomes consisting of endogenous cargo are administered in an amount effective to reduce de novo lipogenesis in the subject.

3. The method of claim 1, wherein the fatty liver disease is non-alcoholic fatty liver disease (NAFLD).

4. The method of claim 3, wherein the subject is suffering from non-alcoholic fatty liver disease (NAFLD).

5. The method of claim 3, wherein the NAFLD is simple fatty liver disease or nonalcoholic steatohepatitis (NASH).

6. The method of claim 1, wherein the intact bovine milk-derived exosomes consisting of endogenous cargo are administered orally.

7. The method of claim 1, wherein the intact bovine milk-derived exosomes consisting of endogenous cargo are administered to the subject in a nutritional composition.

8. The method of claim 7, wherein the nutritional composition is in the form of a powder.

9. The method of claim 7, wherein the nutritional composition is in the form of a liquid.

10. The method of claim 1, wherein the nutritional composition further comprises protein, carbohydrate, and/or a fat.

11. The method of claim 10, wherein the protein comprises whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hamp proteins, flax seed proteins, earthworm protein, insect protein, or combinations of two or more thereof.

12. The method of claim 10, wherein the carbohydrate comprises human milk oligosaccharides (HMOs), maltodextrin, hydrolyzed starch, glucose polymers, corn syrup, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, inulin, fructooligosaccharides, or combinations of two or more thereof.

13. The method of claim 1, wherein fat comprises coconut oil, fractionated coconut oil, soy oil, corn oil, olive oil, safflower oil, medium chain triglyceride oil (MCT oil), high gamma linolenic (GLA) safflower oil, sunflower oil, palm oil, palm kernel oil, palm olein, canola oil, marine oils, fish oils, algal oils, borage oil, cottonseed oil, fungal oils, omega-3 fatty acid, interesterified oils, transesterified oils, structured lipids, or combinations of two or more thereof.

14. The method of claim 13, wherein the fat comprises at least one omega-3 fatty acid selected from the group consisting of eicosapentaenoic acid, docosahexaenoic acid, arachidonic acid, and alpha-linolenic acid.

15. The method of claim 1, wherein the nutritional composition comprises protein, carbohydrate, fat, and one or more nutrients selected from the group consisting of vitamins, minerals, and trace minerals.

16. The method of claim 1, wherein the nutritional composition comprises about 0.001 to about 10 wt % of the intact bovine milk-derived exosomes consisting of endogenous cargo, based on the weight of the nutritional composition.

17. The method claim 1, wherein the subject is a human.
